(19) United States
(12) Patent Application Publication
(10) Pub. No.: US 2026/0083304 A1
(43) Pub. Date: Mar. 26, 2026

(54) ENDOSCOPE WITH A BENDING SECTION HAVING HINGES

(71) Applicant: AMBU A/S, Ballerup (DK)

(72) Inventors: Frederik Clausager Vemb HANSEN, Copenhagen NV (DK); Lasse Markworth JOHNSEN, Birkerød (DK)

(73) Assignee: AMBU A/S, Ballerup (DK)

(21) Appl. No.: 19/337,189

(22) Filed: Sep. 23, 2025

(30) Foreign Application Priority Data
Sep. 25, 2024 (EP) 24202543.5

(51) Int. Cl. A61B 1/008; A61B 1/00; A61B 1/04

(52) U.S. Cl. CPC A61B 1/008; A61B 1/00066; A61B 1/00114; A61B 1/042

(57) ABSTRACT

An endoscope (1) including a bending section (20) molded in one piece and including segments (22) connected to each other by hinges (24). An exterior surface (33) of the segments forms an outer surface of the bending section. A central passage (12) extends through each segment from a distal end to a proximal end of the bending section. Each segment has a recess (18) extending from the exterior surface into the segment. The recesses form a channel (30) extending in parallel with the central passage through the segments. An intermediate hinge and a periphery hinge connect two neighboring segments, where the intermediate hinge is placed between the central passage and the channel, and the periphery hinge is placed at the exterior surface of the segments. Electrical cables (7) are extending between the distal tip and the handle are arranged in the channel.

### CROSS-REFERENCE TO RELATED APPLICATIONS

This application claims priority from and the benefit of European Patent Application No. 24202543.5, filed Sep. 25, 2024; the disclosure of said application is incorporated by reference herein in its entirety.

### TECHNICAL FIELD

The present disclosure relates to an endoscope comprising an insertion cord including a bending section. The bending section is provided with segments interconnected by bendable hinges.

### BACKGROUND

Flexible endoscopes for medical purposes are often provided with a bending section at a distal end of an insertion tube. This enables the user of the endoscope to maneuver the distal tip of the endoscope inside the human anatomy, such as in the airways, in the kidneys or the gastro-intestinal system, and e.g., to study or perform procedures at tissue of interest. The bending section is typically bent by pulling steering wires. If the endoscope is a 2-way bending endoscope, i.e., bending in two opposite directions in the same plane, the bending section will typically be controlled by two steering wires, which are controlled by one steering wire actuator arranged in the handle of the endoscope, allowing the user to bend the bending section by adjusting a bending lever. If the endoscope is a four-way bending endoscope, i.e., also bending in two opposite directions in a second plane perpendicular to the first mentioned plane, the bending section will typically be controlled by four steering wires, where one steering wire actuator controls two steering wires for bending in the first plane, and the other steering wire actuator controls the two other steering wires for bending in the second plane.

The steering wires will pass from the handle to the proximal end of the bending section inside wire pipes, which are tubes having limited compressibility. Thereby so-called Bowden cables are formed. The wire pipes are at their proximal end connected inside the handle. At the distal end of the wire pipes, they are connected to the proximal end of the bending section, whereas the steering wires continue through the bending section, often in dedicated lumens. The wire pipes are held stationary and translatory movement of the steering wire at the proximal end is transmitted to the distal end as a corresponding translatory movement of the steering wire relative to the wire pipe.

For single use endoscopes, the bending section may be molded in one piece from a polymer material. It is often relevant to provide an insertion cord with the smallest possible outer diameter. Here, the bending section may be a limiting factor. Various factors may be considered when designing a bending section for an endoscope.

One factor is a need for sufficient space for the electrical cables connecting the distal tip and the handle of the endoscope. For a given number of cables, providing more space can enable a reduced bending resistance due to lower frictional forces between the cables during bending. This means that the force which the user has to apply in order to bend the bending section can be lower. Another factor is that a relatively large diameter of the working channel may be preferred. This may allow for larger tools to be applied and for larger flow, e.g. when applying suction through the working channel. Also, another factor is that the design of the bending section, should preferably reduce the risk of kinking of the working channel tube inside the bending section, when the bending section is bent at its maximum bending angle. If the bending section is designed such that the risk of kinking is reduced, it may be possible to apply a thinner wall thickness for the working channel tubing.

### SUMMARY

An object of the present disclosure is to provide an endoscope having a bending section with an optimal compromise between the above-mentioned factors.

In a first aspect, the disclosure provides an endoscope comprising a bending section comprising segments connected to each other by two hinges which are placed opposite one another and adjacent an intermediate passage configured for a working channel tube.

In one embodiment according to the first aspect, the endoscope comprises a handle, and an insertion cord extending from the handle and including a bending section molded in one piece. A camera is arranged in a distal tip distal to the bending section. The bending section comprises:
Segments connected to each other by hinges, where the hinges allow two neighboring segments to bend relative to each other in a bending plane, an exterior surface of the segments forms an outer surface of the bending section.
A passage passing each segment and is extending from a distal end to a proximal end of the bending section, where each segment has a circumferential wall, and the circumferential wall has an inner surface facing and encircling the passage.
A recess extending from the exterior surface of the segments into the segment. The recesses form a channel extending in parallel with the central passage through the segments of the bending section.
An intermediate hinge and a periphery hinge are connecting each pair of neighboring segments, where the intermediate hinge is placed between the central passage and the channel, and the periphery hinge is placed at the exterior surface of the segments.
   wherein electrical cables extending between the distal tip and the handle are arranged in the channel.

Hinges in the bending section molded in one piece of material are parts of the bending section made in dimensions such that the hinges can be bent without plastic deformation of the material.

One effect of this embodiment is that it may enable a smaller outer diameter of the bending section, and at the same time provide a design enabling one or more of: more space for electrical cables, a larger diameter working channel tube, and/or a reduced risk of kinking.

This embodiment may further have the effect of making more space available for the cables so that they are able to slide with a relatively low friction during bending operations of the bending section. This means that the force which the user has to apply in order to bend the bending section can be lower.

The embodiment further has the effect that both hinges will support the working channel and reduce the risk of kinking. Kinking of the working channel has been an issue in the small outer diameter endoscopes having an opening between the working channel lumen and the cable lumen.

In a variation of the present embodiment, lips may be arranged at some or all of the segments. The lips are arranged to extend from a transition between the exterior surface of the segments and the recess, and to follow a circumferential outer periphery of the segment. The lips will thus to some extent cover the channel and reduce the risk of the electrical cables being displaced out of the channel.

In a variation of the present embodiment, at least one segment is provided with a film of material covering the recess. This film is in one piece of material with the bending section, and the film has a thickness smaller than a thickness of a hinge. Such a film on one or more segments has the effect of keeping the electrical cables in place in the channel both during manufacturing, but also during maneuvering the endoscope when an endoscopy procedure is performed.

In a second aspect the disclosure relates to a method for assembling an endoscope, comprising:
providing a bending section molded in one piece from a fused polymer, and having:
   Segments are connected to each other by hinges, and the hinges allow two neighboring segments to bend relative to each other in a bending plane. An exterior surface of the segments forms an outer surface of the bending section.
   A passage passing each segment and extending from a distal end to a proximal end of the bending section, where each segment has a circumferential wall, and the circumferential wall has an inner surface facing and encircling the passage.
   A recess extending from the exterior surface of the segments into the segments, forming a channel extending in parallel with the central passage through the segments of the bending section.
   An intermediate hinge and a periphery hinge are connecting each pair of neighboring segments, the intermediate hinge is placed between the central passage and the channel, and the periphery hinge is placed at the exterior surface of the segments.
arranging a working channel tube in the passage,
arranging electrical cables in the channel,
arranging a bending cover on the outer surface of the bending section.

This method has been found to be a cost-effective way to manufacture bending sections for endoscopes, e.g., single-use endoscopes, having a small diameter insertion cord. The outer diameter of the insertion cord, including the bending section, may be 3 mm or smaller. The method may also be relevant for larger outer diameters, such as 4-6 mm, where advantages such as more space for electrical cables, a larger diameter working channel tube, and/or a reduced risk of kinking of the working channel tube, may be achieved.

In a third aspect the disclosure relates to a system comprising an endoscope according to the first aspect and variations thereof, a monitor and a control unit.

Segments of the bending section include a proximal end segment, a distal end segment and intermediate segments arranged between the proximal end segment and the distal end segment. The segments are not necessarily identical. Especially, the proximal end segment and the distal end segment may have features enabling the connection of the bending section to the insertion tube and to the distal tip housing, respectively.

### BRIEF DESCRIPTION OF THE FIGURES

The above-mentioned embodiments and variations, features and advantages thereof will be further elucidated by the following illustrative and nonlimiting detailed description of embodiments disclosed herein with reference to the appended drawings, wherein:

FIG. 1 shows an endoscope and a monitor with a control unit.

FIG. 2 shows an example of a steering wire actuator, here in the form of a roller, and a wire pipe fastener.

FIG. 3A shows a distal end of an endoscope including a bending section.

FIG. 3B shows an enlarged view of part of the bending section in FIG. 3A.

FIG. 4 shows a close-up of the bending section in FIG. 3A viewed from a different angle.

FIG. 5 shows a further close-up of the bending section in FIG. 3A viewed from a different angle.

FIG. 6 shows a cross-sectional view of a segment of a bending section.

FIG. 7 shows a cross-sectional view of a bending section between two segments.

FIG. 8 shows a view similar to FIG. 6, where steering wires, electrical cables and bending cover have been added.

FIG. 9A and FIG. 9B shows two different segments each with a lip extending over the channel, but from opposite sides.

FIG. 10 shows a cross-sectional view of a segment provided with a thin film covering the recess.

In the drawings, corresponding reference characters indicate corresponding parts, functions, and features throughout the several views. The drawings are not necessarily to scale, and certain features may be exaggerated in order to better illustrate and explain the disclosed embodiments. For simplicity, this disclosure will focus on a two-way bending endoscope, but the disclosure is relevant for, and covers, also a four-way bending endoscope.

### DETAILED DESCRIPTION

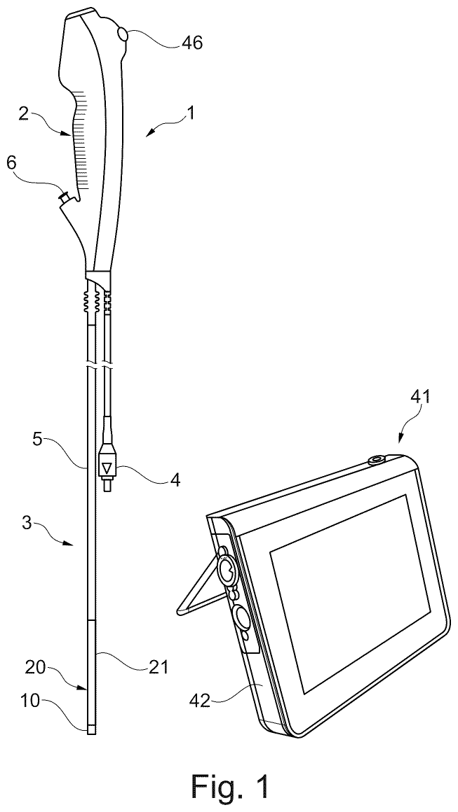

FIG. 1 illustrates an endoscope 1, which comprises a handle 2, an insertion cord 3 and an electrical cable with a connector 4 to connect the endoscope 1 to a monitor 41. The insertion cord 3 is the part to be inserted into a body lumen during an endoscopic procedure. The insertion cord comprises a distal tip 10, a bending section 20 and a main tube 5.

The bending section 20 is covered by a sleeve, or bending cover, 21. The handle 2 may comprise an entrance to a working channel 6 running through the insertion cord to the distal tip. The handle 2 also comprises a bending lever 46, which can be used to bend the bending section 20. The endoscope handle 2 may be considered as an interface from which the insertion cord 3 extends, and which is configured to control a position of the insertion cord 3. The handle 2 or interface may e.g. be detachably connected to e.g. a robotic arm.

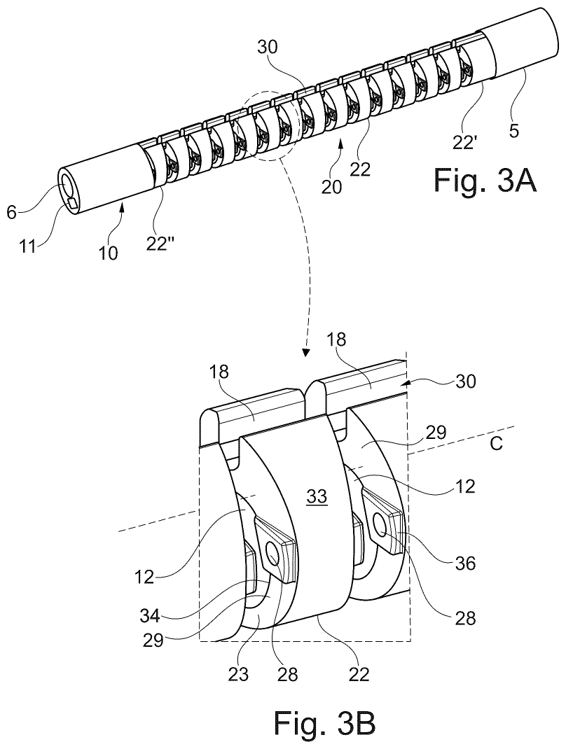

The distal tip 10 comprises housing enclosing a camera 11 (see FIG. 3A) and light emitters (not shown), e.g., in the form of one or more LEDs or the end of an optical light fiber.

The monitor 41 may comprise a control unit 42 including an electronic circuit for receiving and processing an image stream from the camera 11. The electronic circuit may include a processor and memory and may have embedded therein, in an FPGA or the memory, for example, processing instructions configured to perform image processing, provide a graphical user interface, manage storage of images, etc. The control unit 42 may also be provided separately from the monitor 41 and may be communicatively connected thereto. The monitor 41 may comprise a display supported by a housing, as shown. The control unit 42 may be housed in a housing and be communicatively connected to a remote display.

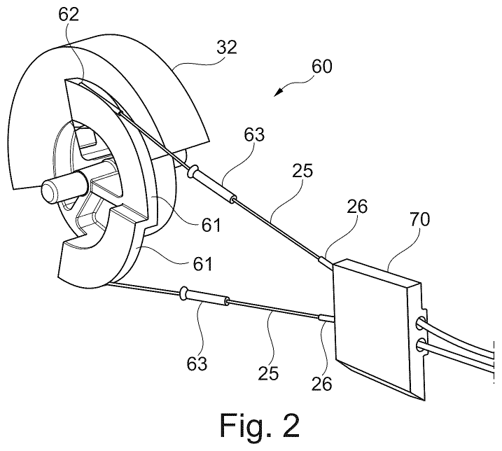

FIG. 2 shows a schematic example of a roller 32 of a steering wire actuator 60 configured to bend the bending section 20 by pulling the steering wires 25. The steering wires 25 are moved by rotation of wire drum curved surfaces 61, which are a circular arc surface or a curved surface placed on the roller 32 and supporting the steering wires 25. In the example, one end of the steering wire 25 has been drawn through a fixing structure 62 and bent back and fastened to itself by a crimp 63. The proximal ends of the wire pipes 26 are fixated in the wire pipe fastener 70, whereas the distal ends of the wire pipes 26 are secured to the proximal end of the bending section 20. Both the wire pipe fastener 70 and the roller 32 are often secured in the handle 2. The system in FIG. 2 is for a two-way bending endoscope.

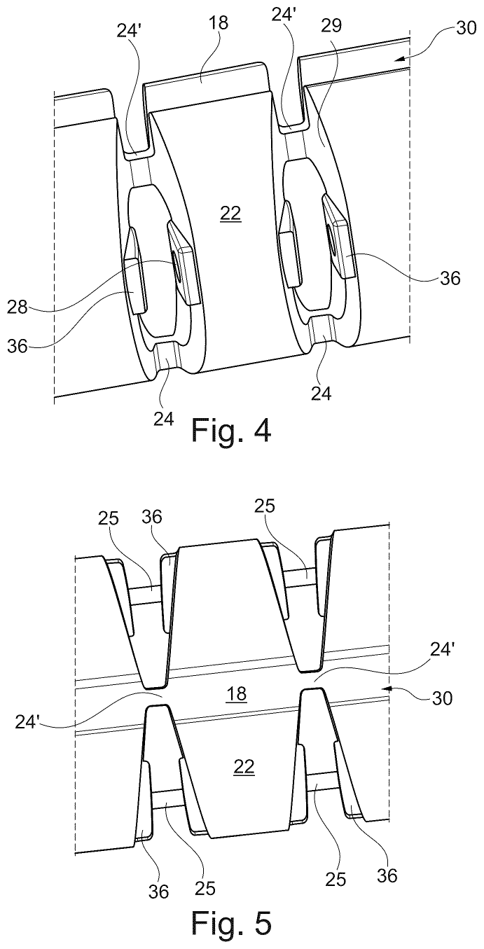

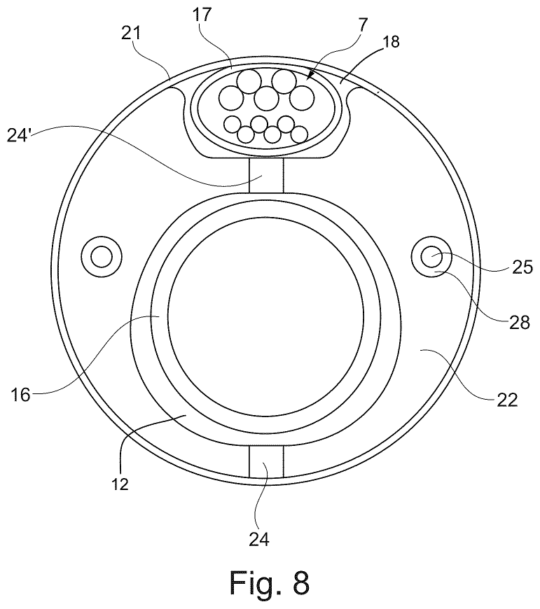

FIG. 3A shows a distal end of the insertion cord 3 of the endoscope 1, where the bending cover 21 (shown in FIG. 8), typically covering the bending section 20 and often making this part watertight, has been removed. The proximal end of the bending section 20 is attached to the main tube 5, and the distal end of the bending section 20 is attached to the distal tip 10. In this example the bending section 20 is molded in one piece and comprises a number of segments 22. A proximal end segment 22′ is connected to the distal end of the main tube 5. A distal end segment 22″ is connected to the distal tip 10. The segments are held together, and interconnecte, by hinges 24, 24′ (see FIG. 4), so that the segments can be reoriented relative to each other by translation of the steering wires 25.

Refering to FIG. 3B, steering wires 25 are guided in steering wire passages 28 and are connected in a fixed connection to the distal end, e.g., the distal end segment 22″ or the tip part. The steering wire 25 is preferably not translatable in relation to the distal end segment 22″. Between the handle and the distal end of the main tube 5, or the proximal end of the bending section 20, the steering wire 25 is guided in the wire pipe 26, which is secured by the wire pipe fastener 70 to the handle 2 distally of the steering wire actuator 60.

The bending section comprises a central passage 12 extending in parallel with a longitudinal center axis C (see FIG. 3B) of the bending section. The center axis C extends in the direction between the proximal end of the bending section and the distal end of the bending section. A working channel tube 16 (see FIG. 8) may pass through the central passage. The center axis C may be placed in the central passage 12 but not necessarily in the center of the central passage 12.

FIGS. 3A and 3B further show that the bending section 20 comprises a channel 30 formed by a number of recesses 18. As shown in FIG. 3B, a segment 22 comprises a circumferential wall 23 with an exterior surface 33 and an inner surface 34 radially opposite the exterior surface 22 and encircling a portion of the central passage 12, lateral surfaces 29 extending between and connecting the exterior surface 33 and the inner surface 34, a recess 18 extending from the exterior surface 33 into the circumferential wall 23 toward the central passage 12, a steering wire passage 28, and bending limiting protrusions 36 extending longitudinally from the lateral surfaces 29 toward an opposing lateral surface of an adjacent segment. When the bending section 20 is straight the channel 30 is straight and parallel with the central passage 12 through the segments 22, 22′, 22″ of the bending section 20. This channel 30 used to receive the electrical cables 7 connecting the camera 11 and the light source in the distal tip 10 with a printed circuit board (PCB) in the handle 2. The electrical cables 7 may be arranged as a bundle, which may be surrounded by a sleeve 17 (see FIG. 8).

FIG. 4 shows an enlarged view of a few segments 22 seen from a different angle than the view in FIG. 3B. In FIG. 4 it is possible to see also the hinges 24, 24′ connecting two neighboring segments 22. Between two segments 22 there is one periphery hinge 24 and one intermediate hinge 24′. The intermediate hinges 24′ are arranged between the central passage 12 and the channel 30. Therefore, the intermediate hinges 24′ are recessed from the exterior surface 33. The periphery hinges 24 extend from the central passage 12 and the exterior surface 33 without being recessed. As indicated in FIG. 4, the thickness of the hinges may be similar to the adjacent thickness of the circumferential wall 23 of the segment 22, in a direction radial to the center axis C. The number of hinges between two segments may be exactly two.

FIG. 5 shows a view of a few segments 22, where the bending section 20 is rotated approximately 90 degrees in relation to the view in FIG. 4. In FIG. 5 the viewing direction is perpendicular to the center axis C and directly into the channel 30 and the intermediate hinges 24′. FIG. 5 also shows the steering wires 25 traversing the bending limiting protrusions 36. A bottom surface of the channel 30 is visible, which comprises bottom surfaces of the recesses 18 (the radially inwardmost surfaces) and the radially outward surfaces of the intermediate hinges 24′.

The bending limiting protrusions 36, which may surround each opening to the steering wire passage 28 in each segment 22, protrude from the lateral surfaces 29 of each segment 22 and face corresponding bending limiting protrusions 36 of the adjacent segments 22, i.e., the surfaces extending perpendicular to the center axis C. The bending limiting protrusions 36 protrude in a direction parallel to the center axis C. The purpose of the bending limiting protrusions 36 is to define the maximum bending angle of the bending section 20 and to avoid that two neighboring segments 22 squeeze the bending cover 21 between them during bending. Such squeezing of the bending cover 21 could result in puncture of the bending cover 21. The bending limiting protrusions 36 can be placed closer to the hinges, can be configured such that the steering wires extend radially distally from the bending limiting protrusions 36, and can be omitted altogether. As shown, the bending limiting protrusions 36, advantageously, also prevent rubbing of the steering wires with the bending cover 21 when the bending section is maximally bent so that the bending limiting protrusions 36 abut each other.

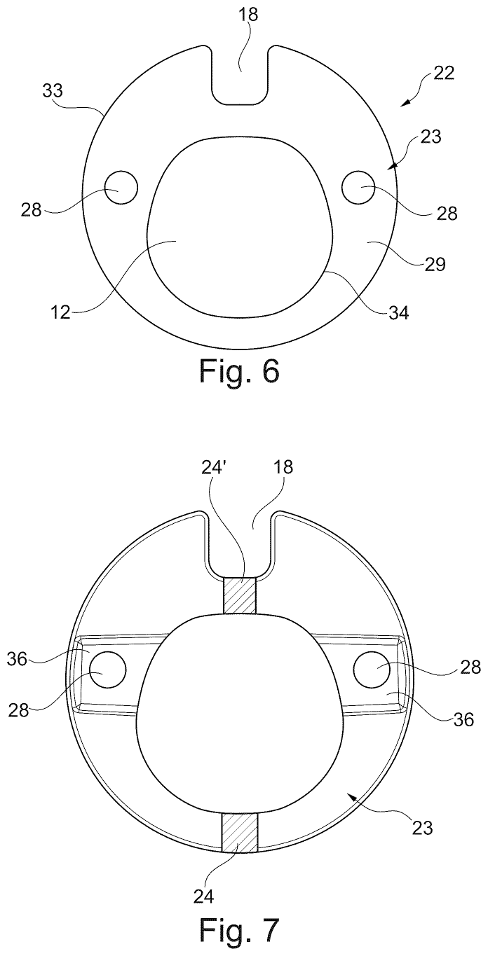

FIG. 6 shows a cross-sectional view of a segment 22 orthogonal to the center axis. The segment 22 comprises an opening for the central passage 12. The central passage 12 passes all segments 22, 22′, 22″ and extends from a distal end to a proximal end of the bending section 20. The circumferential wall 23 of each, or most, segments comprises the inner surface 34 facing and encircling the passage 12, the exterior surface 33 encircling the whole segment 22, the steering wire passages 28 with openings in the lateral surfaces 29, and the recess 18. The recess 18 extends from the exterior surface 33 into the circumferential wall 23 toward the inner surface 34, without reaching the inner surface 34. The recesses 18 will thereby form the channel 30 extending in parallel with the central passage 12 through the segments of the bending section.

FIG. 7 shows a cross-sectional view of a bending section between two segments, orthogonal to the center axis and traversing the hinges 24,24′. This view only intersects the hinges, as opposed to FIG. 6 intersecting the circumferential walls 23 of the segments. The circumferential wall 23 may have a varying wall thickness, where the wall thickness is measured in a radial direction in relation to the center axis C as a distance between the inner surface 34 and the exterior surface 33. This wall thickness may be minimum at a position connected to a hinge 24. Here the wall thickness may be similar to the corresponding thickness of the hinge 24. Thereby, the bottom of the channel 30 may be a plane surface, both where the channel 30 passes the recess 18 of a segment 22 and where the channel 30 passes over a hinge 24. The thickness of the circumferential wall 23 may have a maximum at a position neighboring a recess 18. The cross-sectional view through the hinges 24, 24′ exposes the bending limiting protrusions 36, surrounding the openings for the steering wire passages 28. FIG. 7 shows that the thickness, or height, of the hinges 24,24′ in a radial direction to the center axis C, between the exterior surface 23 and the interior surface 34 or the bottom surface of the recess 18, may be similar or equal to the wall thickness of the portion of the circumferential wall 23 adjacent each hinge. By similar it is meant that the thickness or hinge height may be +/−20% of the wall thickness.

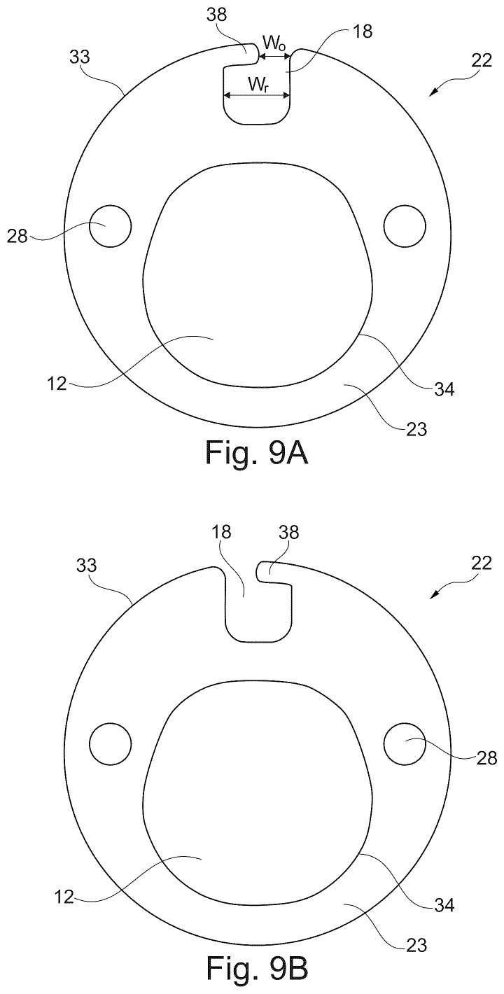

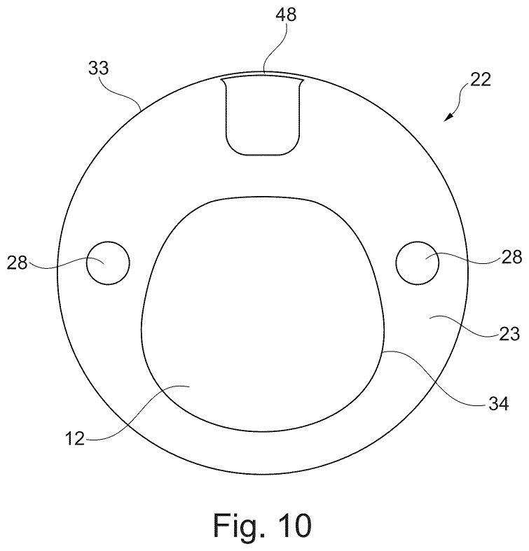

FIG. 8 shows a cross-sectional view through a segment 22 of the bending section 20, where the working channel tube 16, steering wires 25, electrical cables 7 and the bending cover 21 has been added. The steering wire passages 28 are shown as closed passages with a circular cross-sectional shape. Other shapes are also possible, and the steering wire passages 28 may also be open passages in open connection with the central passage 12 for the working channel tube 16, e.g. an opening of the steering wire passage 28 is also an opening in the periphery of the central passage 12, so that the central passage and the steering wire passages form one periphery. Open steering wire passages 28 are disclosed in European Patent Application No. 3925513A1 (see e.g. FIGS. 9 and 10), which is incorporated herein by reference in its entirety.

As shown in FIG. 8, the electrical cables 7 may be arranged in a sleeve 17. The bending cover will keep this sleeve 17 with electrical cables 7 in place during bending and manipulation of the endoscope. The assembling of the endoscope may be simpler when the electrical cables 7 are assembled in a sleeve 17 keeping the cables together.

The shape of the segment 22 may provide support for the working channel tube 16 along the full periphery of the tube. This is opposed to solutions where one larger lumen is applied for both the working channel tube 16 and the electrical cables 7. This further support for the working channel tube 16 will reduce the risk of the tube kinking during bending of the bending section. This means that the maximum bending angle of the bending section may be increased, or the bending radius can be decreased at the same bending angle. Also, the wall thickness of the tube material may be reduced, or a softer (more flexible) material may be used for the working channel tube 16.

Both FIG. 7 and FIG. 8 indicate that the periphery hinge 24 and the intermediate hinge 24′ have similar cross-sectional dimensions. However, in some variations the intermediate hinge 24′ may be higher (thicker) than the periphery hinge 24. The height is here measured in a radial direction from the center axis C. A higher intermediate hinge 24′ may improve the overall torsional stiffness of the bending section 20. This may compensate for the reduction in torsional stiffness caused by having the intermediate hinge 24′ placed closer to the center axis C, compared to a hinge at the outer periphery of the bending section. In an example, the intermediate hinge 24′ has a height which is at least 25 % larger than the height of the periphery hinge 24. The intermediate hinge 24′ may also have a height which is at least 50 % larger than the height of the periphery hinge 24.

It is shown in FIG. 8 that the lumen through the segments for the working channel tube 16 may not have a circular cross-section. This enables having an outer diameter of the working channel tube 16 which is as large as possible, and at the same time still being able to take up variations in the outer diameter of the working channel tube caused by the tolerances on the tube. This means that tubes having the maximum diameter possible within a given tolerance range, can fit into the lumen but may end up having a shape deviating slightly from a circular shape. This is described further in European Patent Application No. 4233679A1 (see e.g. paragraphs [0018] and [0062]), which is incorporated herein by reference in its entirety.

As illustrated, the shape of the recesses 18 in the segments differs between the example shown in FIGS. 6 and 7, and the example shown in FIG. 8. The recess 18 may have different shapes and sizes depending on the outer diameter of the bending section 20 and depending on the number of electrical cables 7 necessary for the specific endoscope. The number of electrical cables 7 depend on the type of image sensor in the distal tip 10, and also on the presence of other transducers in the distal tip. Other transducers could for example be temperature or pressure sensors or an ultrasonic transducer. The shape and size of the recess 18 may also, as mentioned above, depend on the need for reducing friction during bending.

For the recess 18 a cross-sectional area A_{R} may be defined. This is the area limited by the inner surface of the bending cover 21, when the bending cover follows a circular shape around the segments 22, and the exterior surface of the segment. Also, a cross-sectional area A_{W} may be defined for the electrical cables 7. This area A_{W} includes the insulation and any shielding of the cables, as well as any sleeve 17 for the electrical cables 7. The relationship between the two areas may have an impact on the friction between the cables 7 and the friction between the cables and the segments 22, when bending the bending section 20, and thereby the bending resistance may also be impacted by this relationship. In one variation the cross-sectional area of the cables A_{W} is in the range 10 % to 85 % of the cross-sectional area of the recess A_{R}. In one variation A_{W} is in the range 10 % to 65 % of A_{R}. In one variation A_{W} is in the range 10 % to 50 % of A_{R}.

FIGS. 9A and 9B show two different segments, each with a lip, or circumferential protrusion, 38, extending over the recess 18. In FIG. 9A the lip 38 extends from the left side of the recess in the cross-sectional view, and in FIG. 9B the lip 38 extends from the right side of the recess in the cross-sectional view. These lips have the effect of keeping the electrical cables 7 inside the channel 30 during bending of the bending section. The bending cover 21 has the same effect, but displacement of the electrical cables may cause some bulging of the bending cover 21, as the bending cover 21 typically has more elasticity than the bending section material. The lips 38 made in the same material, and in one piece with the bending segments 22, will prevent any bulging.

These lips 38 may further have the effect of holding or supporting the electrical cables 7 or a sleeve 17 enclosing the electrical cables 7, in place during manufacturing of the endoscope 1 in the period from arranging the electrical cables 7 in the channel 30 and until the bending cover 21 has been arranged.

As shown in FIGS. 9A and 9B, the lips 38 have dimensions such that it is still possible to insert a sleeve 17 enclosing the electrical cables 7 through an opening into the channel 30. Also, FIG. 9A shows a different segment 22 of the bending section 20 than FIG. 9B. I.e. in a variation the lips 38 may be connected to opposite sides of the channel 30 for different segments 22. This will reduce the risk that electrical cables 7 may be displaced out of the channel 30 during bending operations.

Lips 38 may be distributed in different patterns along the length direction of the bending section 20. E.g., a lip 38 could be arranged on all or a majority of the segments 22 of the bending section 20. These lips 38 could all extend from the same side of the channel 30, or the lips could extend from one side or from the other side, alternately. A lip 38 could also be arranged on every second or every third or every fourth segment 22 only. Also here, the lips could extend from one side of the channel 30 or from the other side, alternately.

In an example, at least one lip 38 on one segment 22 extends from a first side of the channel 30, and at least one lip 38 on another segment 22 extends from a second, opposite, side of the channel 22. In this example, the two segments 22 mentioned may only be provided with one lip 38 each.

In a variation, a segment may be provided with two lips 38 extending from each side of the recess 18 and covering part of the recess 18 and may follow a circumferential outer periphery of the segment 22. The two lips 38 extending over one recess 18 may have different dimensions and extending over a different length of the recess 18 along a circumferential outer periphery of the segment 22.

The lips 38 may have a thickness in a radial direction relative to the center axis C, so that they are sufficiently flexible to be bent slightly during arrangement of the electrical cables 7 into the channel 30. If the bending section 20 is made from POM this thickness could be in the range 0.05-0.4 mm, or in the range 0.1-0.3 mm. These ranges may also be relevant for polypropylene, but other materials may have other properties in relation to molding capabilities.

The lips 38 may have a length in the direction parallel to the center axis C which is similar to, or equal to the length of the segment 22. The lips 38 may also have a length which is shorter than the length of the segments 22.

The width wᵣ of the recess 18 (see FIG. 9A) forming the channel 30 may be in the range of 0.8-3.0 mm depending on e.g. the outer diameter of the bending section 20 and on the number and diameter of electrical cables 7. When a lip 38 is arranged on a segment 22 to partly extend over the recess 18, the lip 38 will result in an opening into the recess 18 having a width wₒ which may be in the range of 0.3-2.5 mm. In a variation the width of the opening wₒ may be at least 25 % of the width of the recess wᵣ. Also, the width of the opening wₒ may be at least 40 %, or at least 50 %, or at least 60 % of the width of the recess wᵣ. An upper limit may be that the width of the opening wₒ is at maximum 95 % of the width of the recess wᵣ.

A further variation is shown in FIG. 10, where the segments 22 or some of the segments 22, are provided with a thin film 48 of material covering the recess 18. This thin film 48 supports the electrical cables 7 and keeps them in place but is too thin to function as a hinge. The thin film 48 may be in one piece of material and integral with the bending section 20, e.g., molded in one piece with the bending section 20. Molding in one piece may here be understood to include also a 2K molding process, where e.g. different polymers are fused together. The film 48 may have a thickness smaller than a thickness of a hinge 24, e.g. smaller than half the thickness of a hinge 24. The thin film 48 may be overmolded after the remainder of the bending section has been formed.

In an example, the bending section 20 is made from POM and has segments 22 with an outer diameter in the range 3.0-6.5 mm, e.g. 5.5-6.2 mm, in a cross-sectional view, where the cross-section is made perpendicular to the center axis C. In a similar viewing angle, the hinges 24, 24′ have dimensions in the range 0.4×0.4 mm to 0.6×0.6 mm. The height and the width of the hinges 24, 24′ may differ, and the height of the intermediate hinge 24′ may be up to up to 0.8 or 0.9 mm. The width wᵣ of the recess 18 may be in the range 1.5-3.0 mm, e.g. 2.0-2.8 mm.

The height of the channel 30 may be defined in a radial direction relative to the center axis C as the distance between the intermediate hinges 24′ and the bending cover 21, when the bending cover 21 follows a circular cross-sectional shape. This height may be in a range of 0.8-2.0 mm. The thickness of the bending cover 21 may be in the range of 0.05-0.1 mm.

Lips 38 may be arranged to extend over a number of recesses 18 along the length of the channel 30 in the bending section. The lips 38 may be designed to cover 40-60 % of the recess 18. If the width wᵣ of the recess is 2.0 mm, the width of the opening wₒ may be 0.8-1.2 mm. The outer diameter of the individual electrical cables 7 may be in a range of 0.1-0.5 mm, and there may be 3-8 cables, e.g. 5 cables. In some cases, there may be 12-70 electrical cables 7.

The bending section 20 may be injection molded, may be manufactured with a 3D printing machine, or may be molded from polymers in any other way. In an injection molding process embodiment, the central passage 12 may be made with a mandrel. Side-action slides slide into position as the mold, which comprises two parts that together form the circumferential wall surfaces of the bending section 20, come together. When the mold is closed and the mandrels and side-action slides are in position, a melted polymer composition is injected into channels in the mold that are fluidly coupled with an injection molding extruder. The side-action slides form the spaces between the segments and the hinges. The mold may comprise a longitudinal protrusion to form the channel 30 or may comprise a second mandrel to do so. Additional mandrels, or rods, can be used to form the steering wire passages 28. If the steering wire passages are open, a common mandrel can be used to form the steering wire passages and the central passage 12.

The following items are further variations and examples of the embodiments described with reference to the figures.
1. An endoscope comprising: a handle; an insertion cord extending from the handle and including a bending section molded in one piece, and a camera arranged in a distal tip distal to the bending section, the bending section comprising: segments connected to each other by hinges, the hinges allowing two neighboring segments to bend relative to each other in a bending plane, an exterior surface of the segments forms an outer surface of the bending section, a central passage passing through each segment and extending from a distal end to a proximal end of the bending section, where each segment has a circumferential wall, the circumferential wall has an inner surface facing and encircling the central passage, a recess extending from the exterior surface of the segments into the segments, forming a channel extending in parallel with the central passage through the segments of the bending section, an intermediate hinge and a periphery hinge connecting each pair of neighboring segments, the intermediate hinge being located between the central passage and the channel, and the periphery hinge being located at the exterior surface of the segments; wherein one or more electrical cables extending between the distal tip and the handle are arranged in the channel.
2. The endoscope according to item 1, wherein a bending cover is arranged on the outer surface of the bending section and is configured to cover the recess to keep the one or more electrical cables in the channel.
3. The endoscope according to item 1 or 2, wherein lips are arranged on at least two segments, the lips being arranged to extend from a transition between the exterior surface of the segments and the recess, and to follow a circumferential outer periphery of the segment.
4. The endoscope according to item 3, wherein the lips being configured such that the width of an opening to the recess is smaller than a maximum width of the recess.
5. The endoscope according to item 4, wherein at least one lip on one segment is extending from a first side of the recess and at least one lip on another segment is extending from a second opposite side of the recess.
6. The endoscope according to any one of the previous items, wherein the intermediate hinge and the periphery hinge are placed on opposite sides of the central passage.
7. The endoscope according to any one of the previous items, wherein at least one segment has a film of material covering the recess, where the film is in one piece of material with the bending section, and the film has a thickness smaller than a thickness of a hinge.
8. The endoscope according to any one of the previous items, wherein a cross-sectional area of the electrical cables is between 10 % and 85 % of a cross-sectional area of the recess.
9. The endoscope according to any one of items 1-7, wherein the cross-sectional area of the electrical cables is between 10 % and 65 % of the cross-sectional area of the recess.
10. The endoscope according to any one of the previous items, wherein a working channel tube is arranged in the central passage.
11. The endoscope according to any one of the previous items, wherein the number of hinges between two segments is exactly two.
12. The endoscope according to any one of the previous items, wherein the circumferential wall has a varying thickness with a minimum thickness at a position connecting to a hinge, and a maximum thickness next to the recess.
13. The endoscope according to any one of the previous items, wherein the intermediate hinge has a height, measured in a radial direction from a center axis, which is at least 25 % larger than the height of the periphery hinge.
14. A method for assembling an endoscope according to any one of the previous items, comprising: providing a bending section molded in one piece from a fused polymer, and having: segments connected to each other by hinges, the hinges allowing two neighboring segments to bend relative to each other in a bending plane, an exterior surface of the segments forms an outer surface of the bending section, a passage passing each segment and extending from a distal end to a proximal end of the bending section, where each segment has a circumferential wall, the circumferential wall has an inner surface facing and encircling the passage, a recess extending from the exterior surface of the segments into the segments, forming a channel extending in parallel with the central passage through the segments of the bending section, an intermediate hinge and a periphery hinge are connecting each pair of neighboring segments, the intermediate hinge is placed between the central passage and the channel, and the periphery hinge is placed at the exterior surface of the segments, arranging a working channel tube in the passage, arranging electrical cables in the channel, arranging a bending cover on the outer surface of the bending section.
15. A system comprising an endoscope according to any one of items 1-13, a monitor and a control unit.

The term “distal” is defined to be in the direction toward the patient, and the term “proximal” is defined to be in the direction away from the patient. For the handle of the endoscope, the distal end will be the end where the insertion tube is connected, and the proximal end is the opposite end. Further, the expression “handle” may be a positioning interface, or interface, which functions to control the position of the insertion cord and operating the bending section. The handle, or positioning interface, may be an interface operated by a robotic arm, or it may be a handle operated by the hand of an endoscope user.

In the present disclosure, one steering wire is counted as a part extending from the steering wire actuator (or roller) to the distal end of the bending section. If an element extends from the steering wire actuator to the distal end of the bending section and back to the steering wire actuator it is counted as two steering wires and may be referred to as first and second steering wires. Thus, two steering wires may comprise distinct elements without an intermediate part connecting them at the distal end or parts of one element that includes an intermediate part connecting them at the distal end. A steering wire may comprise one string or a bundle of strings. The strings may be comprised or metal or polymer.

In the present disclosure, an electrical cable comprises an insulated conductor. The conductor may comprise one strand or multiple strands. The electrical insulator may be coated on the conductor or may comprise a sheath or a sleeve surrounding the conductor. A pair of cables may comprise a coaxial cable or a twisted pair cable or an untwisted pair cable. Two or more cables may be surrounded by a shield or a sleeve.

The use of the terms “first”, “second”, “third”, “fourth”, “primary”, “secondary”, “tertiary” etc. does not imply any particular order or importance. These labels are included to identify individual elements. Furthermore, the labelling of a first element does not imply the presence of a second element and vice versa.

As used herein, “in the range” includes the values that define the range. Therefore, “in the range of A-B”includes A and B.

### LIST OF REFERENCES

1 endoscope
2 handle
3 insertion cord
4 electrical cable with connector
5 main tube
6 working channel
7 electrical cables
10 distal tip
11 camera
12 central passage
16 working channel tube
17 sleeve for electrical cables
18 recess
20 bending section
21 bending cover
22 segment
22′ proximal end segment
22″ distal end segment
22′ proximal end segment
22″ distal end segment
23 circumferential wall
24 periphery hinge
24′ intermediate hinge
25 steering wire
26 wire pipe
28 steering wire passage
29 lateral surface of the circumferential wall
30 channel
32 roller
33 exterior surface
34 inner surface
36 bending limiting protrusion
38 lip
41 monitor
42 control unit
46 bending lever
48 thin film
60 steering wire actuator
61 wire drum curved surface
62 fixing structure
63 crimp
70 wire pipe fastener
C center axis
wᵣ width of recess
wₒ width of opening to recess

1. An endoscope comprising:
a handle;
an insertion cord extending from the handle and including a bending section and a distal tip, the distal tip extending from a distal end of the bending section and comprising a camera; and
one or more electrical cables extending between the distal tip and the handle,
wherein the bending section comprises a one-piece part including:
a central passage extending from a proximal end to a distal end of the bending section,
segments comprising circumferential walls, the circumferential walls including exterior surfaces, inner surfaces and recesses, the inner surfaces facing the central passage, the exterior surfaces forming an outer surface of the bending section, and the recesses extending longitudinally from the exterior surfaces into the segments and forming a channel extending in parallel with the central passage, and
hinges interconnecting the segments and configured to enable the bending section to bend in a bending plane, the hinges comprising intermediate hinges and periphery hinges, each of the intermediate hinges located radially between the central passage and the channel, and each of the periphery hinges located radially between the central passage and the exterior surface of the respective segment, and
wherein the one or more electrical cables are positioned in the channel.

2. The endoscope of claim 1, wherein the circumferential wall comprises lateral surfaces, wherein the recesses comprise longitudinal slots extending between and connecting the lateral surfaces, each of the longitudinal slots comprising an exterior opening in the exterior surface and a bottom surface opposite the exterior opening, the bottom surface forming an exterior surface of a respective intermediate hinge.

3. The endoscope of claim 2, wherein at least two of the segments comprise lips configured to overlap in part the bottom surfaces of the respective recesses.

4. The endoscope of claim 3, wherein the exterior opening of each of the at least two of the segments comprises an exterior opening width measured perpendicularly to the channel, wherein each of the at least two of the segments comprises a maximum opening width measured perpendicularly to the channel, and wherein the exterior opening width is smaller than the maximum opening width.

5. The endoscope of claim 1, further comprising a bending cover arranged on the outer surface of the bending section and configured to cover the recesses to keep the one or more electrical cables in the channel.

6. The endoscope of claim 5, wherein at least two of the segments comprise lips arranged to extend from a transition between the exterior surfaces and the recesses of the at least two of the segments, and wherein the lips follow a circumferential outer periphery of the circumferential walls of the at least two of the segments.

7. The endoscope of claim 6, wherein each of the at least two of the segments comprises an exterior opening width measured perpendicularly to the channel, wherein each of the recesses comprises a maximum opening width measured perpendicularly to the channel, and wherein the lips are configured such that the exterior opening width of the respective recess is smaller than a maximum width of the recess, the maximum width measured perpendicularly to the channel.

8. The endoscope of claim 1, wherein the intermediate hinges and the periphery hinges are located on opposite sides of the central passage.

9. The endoscope of claim 1, wherein at least two of the segments comprise lips arranged to extend from a transition between the exterior surfaces and the recesses of the at least two of the segments, wherein the lips follow a circumferential outer periphery of the circumferential walls of the at least two of the segments, and wherein the lips are configured such that the width of an opening to the recess is smaller than a maximum width of the recess.

10. The endoscope of claim 9, wherein one of the lips extends from a first side of a respective recess and another of the lips extends from a second side of a respective recess, the first side being opposite the second side and the channel extending between the first side and the second side.

11. The endoscope of claim 1, wherein the recesses comprise longitudinal slots, each of the longitudinal slots comprising an exterior opening in the exterior surface and longitudinally extending surfaces intermediate the exterior opening and a respective intermediate hinge, the longitudinally extending surfaces facing each other, and wherein at least two of the segments comprise lips protruding from one or the other of the longitudinally extending surfaces.

12. The endoscope of claim 11, wherein a maximum distance between the longitudinally extending surfaces is greater than a maximum width of the exterior openings of the at least two of the segments.

13. The endoscope of claim 11, wherein the longitudinally extending surfaces comprise a first surface and a second surface, and wherein the maximum width of the exterior openings of the at least two of the segments extends between the first surface and one of the lips or between the second surface and the one of the lips.

14. The endoscope of claim 11, wherein the longitudinally extending surfaces comprise a first surface and a second surface, wherein the lips comprise a first lip and a second lip, wherein the first lip extends from the first surface toward the second surface, and wherein the second lip extends from the second surface toward the first surface.

15. The endoscope of claim 1, wherein at least two of the segments comprise lips arranged to extend from a transition between the exterior surfaces and the recesses of the at least two of the segments, and wherein the lips follow a circumferential outer periphery of the circumferential walls of the at least two of the segments.

16. The endoscope of claim 1, wherein one of the segments comprises a film of material covering the respective recess, wherein the film is in one piece of material with the bending section, and wherein the film has a thickness smaller than a thickness of a hinge of the hinges.

17. The endoscope of claim 1, wherein a cross-sectional area of the electrical cables is between 10 % and 85 % of a cross-sectional area of one of the recesses.

18. The endoscope of claim 1, wherein each of the circumferential walls comprises a varying wall thickness with a minimum thickness at a position connecting to a peripheral hinge and a maximum thickness next to the recess of the circumferential wall.

19. The endoscope of claim 1, wherein each of the periphery hinges has a height, and wherein each of the intermediate hinges has a height, measured in a radial direction from a center axis, which is at least 25 % larger than the height of the periphery hinge.

20. A system comprising:
the endoscope of claim 1;
a monitor; and
a control unit.
